(19) United States
(12) Patent Application Publication
(10) Pub. No.: US 2009/0131746 A1
(43) Pub. Date: May 21, 2009

(54) CAPSULE ENDOSCOPE SYSTEM AND METHOD OF PROCESSING IMAGE DATA THEREOF

(75) Inventors: Young Dae Seo, Daejeon (KR); Han Bo Shim, Seongnam-si (KR); Dong Ha Lee, Anyang-si (KR)

Correspondence Address: Workman Nydegger;1000 Eagle Gate Tower, 60 East South Temple, Salt Lake City, UT, 84111, US

(73) Assignee: INTROMEDIC CO., LTD., Seoul (KR)

(21) Appl. No.: 12/099,721

(22) Filed: Apr. 8, 2008

(30) Foreign Application Priority Data
Nov. 15, 2007 (KR) 10-2007-0116702

(51) Int. Cl. A61B 1/00; G06K 9/00

(52) U.S. Cl. 600/101; 600/118; 382/128

(57) ABSTRACT

The present invention relates to a capsule endoscope system and method of processing image data thereof and is suitable for capturing the inner parts of human body using a capsule endoscope having an internally-usable capsule size and receiving to display image data generated from the capturing. By the present invention, a possible-disease part of a patient is selected and observed through a number of image data received from a capsule endoscope, attribute information containing diagnostic details are supported to facilitate an overall diagnostic result to be interpreted, and internal organs in numerous image data received from a capsule endoscope are easily discriminated from each other.

This application claims the benefit of the Korean Patent Application No. 10-2007-0116702, filed on Nov. 15, 2007, which is hereby incorporated by reference as if fully set forth herein.

### BACKGROUND OF THE INVENTION

1. Field of the Invention

The present invention relates to a capsule endoscope system, and more particularly, to a capsule endoscope system and method of processing image data thereof. Although the present invention is suitable for a wide scope of applications, it is particularly suitable for capturing the inner parts of human body using a capsule endoscope having an internally-usable capsule size and receiving to display image data generated from the capturing.

2. Discussion of the Related Art

Generally, an endoscope is an insertion type medical instrument devised to observe internal organs for medical examination of diseases without surgery or autopsy.

Unfortunately, the endoscope causes pain and unpleasantness accompanying with the examination to a patient. So, most of patients wish to have medication instead of the endoscope checkup. Specifically, although endoscopes have been used for various diagnoses and checkups, patients and medical staffs have suffered from various inconveniences due to sizes and rigidity of conduit.

Recently, a capsule type endoscope, which complements for various disadvantages of old endoscopes, has been developed and is widely used in diagnosing various diseases for medical fields.

The capsule type endoscope, i.e., a capsule endoscope is an endoscope in an internally-usable capsule size. The capsule endoscope is internally taken through the oral cavity, migrates in accordance with peristalsis of a digestive organ of coeloms to capture an image of the digestive organ, and then transmits the captured image information to an external device by wireless communication.

The capsule endoscope needs no anesthesia without causing nausea and is capable of capture various internal organs including the small intestine that is not captured by a conventional endoscope. Hence, the capsule endoscope enables more precise medical diagnoses.

Image captured by the capsule endoscope is displayed on a monitor. If so, a doctor is able to diagnose a patient's case through the image displayed on the monitor.

The capsule endoscope, which has a considerably small size, captures tens of thousands object images while migrating in accordance with peristalsis of the digestive organ for 8˜10 hours. Endoscope device including the capsule endoscope basically consists of a receiver receiving an image captured by the capsule endoscope by wireless and a workstation including a monitor displaying the image received by the receiver.

As the images received by the receiver are displayed on the monitor, a doctor is able to diagnose a patient's case by checking the images one by one.

In checking tens of thousands images one by one, some of the images correspond to the parts unnecessary for the diagnosis or may not be necessary for the medical examination. Nonetheless, the doctor has to keep watching the entire images displayed on the monitor.

As the doctor diagnoses a patient's case by checking the whole images displayed on the monitor, time is uselessly wasted and it is a time-consuming job.

Meanwhile, in a display type according to a related art, there exist a main display area for displaying received real-time images in accordance with a flow of time and a separate display area for displaying some of the images displayed on the main display area as thumbnails forms. The thumbnail-form display enables a specific disease-possible part to be selectively observed, thereby facilitating interpretation of overall diagnostic result.

However, in the related art, several still images are simultaneously displayed as thumbnails on the separate display area of the monitor screen only. Since the related art fails to support attribute information containing diagnostic details on the still images displayed on the separate display area at all, it is difficult to effectively obtain a diagnosis on a disease-possible part.

Besides, in diagnosing a case of patient through the displayed images, it is important for a doctor to discriminate internal organs from each other. Yet, the related art enables the received image to be displayed on the monitor only but fails to provide any logical tools that facilitate a doctor to discriminate internal organs from each other. Since the doctor should keep an eye on the images to recognize changes of the internal organs while the whole images pass in accordance with a flow of time, the doctor has difficulty in diagnosing a precise disease-possible part.

### SUMMARY OF THE INVENTION

Accordingly, the present invention is directed to a capsule endoscope system and method of processing image data thereof that substantially obviate one or more problems due to limitations and disadvantages of the related art.

An object of the present invention is to provide a capsule endoscope system and method of processing image data thereof, by which a possible-disease part of a patient is selected and observed through a number of image data received from a capsule endoscope.

Another object of the present invention is to provide a capsule endoscope system and method of processing image data thereof, by which attribute information containing diagnostic details are supported to facilitate an overall diagnostic result to be interpreted.

Another object of the present invention is to provide a capsule endoscope system and method of processing image data thereof, by which internal organs in numerous image data received from a capsule endoscope are easily discriminated from each other.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a method of processing image data received from a capsule endoscope by a workstation according to the present invention includes the steps of playing back the received image data on a screen, if a diagnostic result from a played image of the image data is inputted, storing a file corresponding to the mage data by interconnecting the file to the inputted diagnostic result, wherein the diagnostic result is stored as attribute information of the file, creating an image tool indicating the attribute information, displaying the created image tool on the screen, if the displayed image tool is selected, reading the stored file and the stored attribute information, and displaying the read file and the read attribute information on the screen.

In another aspect of the present invention, a capsule endoscope system includes a capsule endoscope generating image data by capturing interior of a digestive organ of a human body by migrating along the digestive organ, the capsule endoscope consecutively transmitting the generated image data using the human body as a conductor, a receiver storing the image data by receiving the image data from a sensor detecting the image data via the human body, and a workstation receiving the stored image data from the receiver, the workstation playing back the received image data, the workstation capturing a portion of the received image data, the workstation storing the captured image data by storing a diagnostic result from the captured image data as attribute information together with the captured image data, the workstation creating an image tool indicating the attribute information and displaying the created image tool on a screen.

Preferably, the workstation includes a first workstation receiving the stored image data from the receiver, the first workstation capturing partial image data from the received image data, the first workstation transmitting the captured image data and a second workstation connected to the first workstation via a network, the second workstation receiving the captured image data from the first workstation, the second workstation storing the diagnostic result as the attribute information of the captured image data together with the received captured image data, the second workstation creating the image tool indicating the attribute information, the second workstation displaying the created image tool on the screen.

More preferably, the second workstation provides a comment input window for performing a comment information input for the captured image data to the screen and the second workstation stores the comment information inputted via the comment input window by interconnecting the comment information to the captured image data.

According to the present invention, numerous image data received from a capsule endoscope are sorted. In storing the sorted image data, attribute information of the image data is interconnected. Image tool for summarizing and representing the attribute information is provided to a screen. Hence, a user, e.g., a doctor is facilitated to confirm patient's symptoms of a disease.

In particular, if the attribute information indicates a diagnostic result by a doctor, when the image data is stored, doctor's comments inputted by the doctor are interconnected to the stored image data. Hence, the present invention facilitates overall diagnostic result of patient to be confirmed and analyzed.

According to the present invention, an image tool indicating an internal organ discriminating image frame in numerous image data received from a capsule endoscope is automatically provided to a screen. And, a function for confirming the internal organ discriminating image frame affirmatively is provided. Hence, a doctor is facilitated to discriminate each internal organ of a digestive organ without viewing playback images of the numerous image data received from the capsule endoscope.

Therefore, the present invention facilitates a doctor to make an accurate diagnosis from numerous image data received from capsule endoscope.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a block diagram of a capsule endoscope system according to one embodiment of the present invention;

FIG. 2 is a flowchart of a process for processing medical image data according to one embodiment of the present invention;

FIGS. 3 to 8 are diagrams for images according to the flowchart shown in FIG. 2;

FIG. 9 is a diagram for a configuration of a capsule endoscope system according to another embodiment of the present invention;

FIG. 10 is a flowchart of a process for processing medical image data according to another embodiment of the present invention;

FIG. 11 and FIG. 12 are diagrams for images according to the flowchart shown in FIG. 10;

FIG. 13 is a diagram of a playback image in an internal organ discriminating mode according to one embodiment of the present invention; and

FIG. 14 is a diagram of a playback image in an internal organ discriminating mode according to another embodiment of the present invention.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

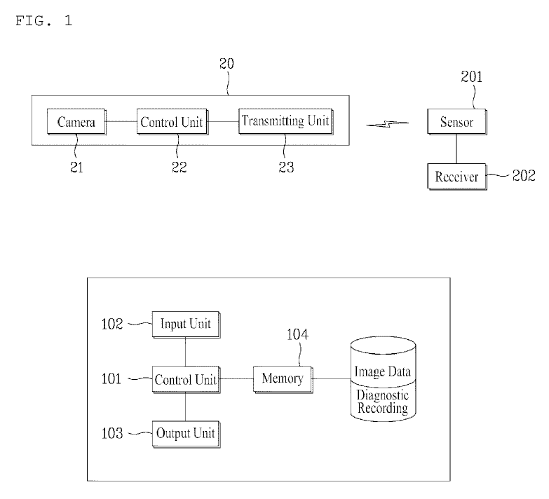

FIG. 1 is a block diagram of a capsule endoscope system according to one embodiment of the present invention, in which a configuration for displaying image data is shown.

Referring to FIG. 1, a system according to the present invention includes a capsule endoscope 20, a receiver 202, and a workstation 100.

The capsule endoscope 20 has a capsule shape and approximately has a radius 11 mmm and a length 24 mm to capture internal organ images of a human body. Once a patient swallows the capsule endoscope 20, the capsule endoscope 20 migrates to the anus from the gullet along a digestive organ for about 10 hours. In the course of the migration, a camera 21 provided within the capsule endoscope 20 takes pictures of the interior of the digestive organ.

The camera 21 of the capsule endoscope 20 includes a ultra-precise lens having a viewing angle of 150° and captures an image of an object through the lens.

The capsule endoscope 20 consecutively transmits image data using a human body conductive system. In particular, the human body plays a role as a conductor in image data transmission of the capsule endoscope 20.

Subsequently, after a current is made to flow through the human body, the image data it transmitted outside the human body to obtain real-time moving pictures (stream images) captured within the human body.

In the aforesaid human body conductive system, image information obtained from capturing the interior of the digestive organ by the capsule endoscope 20 is converted to digital signals of 0 and 1. The converted digital signals are weakly applied as electric signals having positive and negative polarities to the human body.

The electric signal applied to the human body is inputted to the receiver 202 outside the human body and then restored into image data. The restored image data is converted to a playable data format.

In order to input the electric signal to the receiver 202, the system according to the present invention includes a sensor 201 attached to the human body. The sensor 201 is electrically connected to the receiver 202. The sensor 201 detects the electric signal applied to the human body and then forwards the detected electric signal to the receiver 202. And, the capsule endoscope 20 further includes a control unit 22 and a transmitting unit 23 internally as well as the camera 21.

The control unit 22 of the capsule endoscope 20 controls operations of the camera 21 and the transmitting unit 23 and also controls power supply to the camera 21 and the transmitting unit 23. And, the capsule endoscope 20 further includes a low-power battery for the power supply.

Considering the operational control by the control unit 22, the control unit 22 controls the camera 21 to capture an object image inputted via the lens. And, the control unit 22 controls the transmitting unit 23 to externally transmit the image captured by the camera 21.

If the transmitting unit 23 applies an electric signal corresponding to the image forwarded by the control unit 22 to the human body, the sensor 201 attached to the human body detects the electric signal.

The sensor 201 is provided to a sensor pad (not shown in the drawing) to be directly attached to a skin of the human body and connected to the receiver 202 via a cable for the electrical connection to the receiver 202.

Substantially, the capsule endoscope 20 transmits an image in the above-mentioned electric signal form. In the following description, it may be assumed that the capsule endoscope 20 externally transmits the image data itself. The capsule endoscope 20 is provided with a main chip responsible for the functional configuration. And, the main chip includes a communication module according to a low-power communication protocol and a low-power image sensor. The capsule endoscope 20 keeps operating within the human body for 11˜13 hours by the main chip and captures 50,000˜80,000 images corresponding to 100,000 pixels (vertical 320 pixels×horizontal 320 pixels). And, three images are transmitted per a second.

Each time three images are captured per second, the capsule endoscope 20 transmits the captured images to the receiver 202 via the sensor 201 by real time. In this case, the transmitted images correspond to an image stream. Accordingly, the receiver 202 stores medical image data containing at least 118,800 images. The medical image data mean the image data of a whole digestive organ photographed for about 10 hours by the capsule endoscope 20.

The receiver 202 receives the image streams from the capsule endoscope 20 until the image capturing by the capsule endoscope 20 is completed. And, the receiver 202 stores the medical image data. The receiver 202 then transmits the stored medical image data to the workstation 100.

For the data transmission between the receiver 202 and the workstation 100, the receiver 202 and the workstation 100 are connected through USB terminals.

The workstation 100 plays back the medical image data received from the receiver 202 and captures the images in part according to a user's command or automatically in the course of playback. The captured image means the image data captured among the whole medical image data.

The workstation 100 preferably plays back the medical image data by a frame unit. Preferably, the captured image corresponds to the frame unit. In general, a user is a doctor capable of diagnosing disease symptoms. In the following description, the doctor is mentioned as a user as well. So, it is preferably understood that a user is a doctor. Yet, the user is not limited to the doctor. And, the user may include a personnel engaged in a medical facility.

The user inputs a command for capturing images of a part having possible disease symptoms to the workstation 100 while the medical image data received from the receiver 202 are displayed on the monitor. And, the user records comments for the image data captured through the workstation 100.

The workstation 100 includes an input unit 102, a control unit 101, an output unit 103m and a memory 104. The input unit 102 receives medical image data from the receiver 202.

The input unit 102 is provided with a USB port for the reception of the medical image data.

The input unit 102 is provided with a keyboard and mouse for receiving an input of text information about doctor's comments for specific image data and a command for image editing.

The output unit 103 corresponds to a monitor for displaying the medical image data on a screen.

The control unit 101 receives the medical image data received via the input unit 102 and then stores the received medical image data in the memory 104. And, the control unit 101 enables the received medical image data to be displayed through the output unit 103.

The workstation 100 of the present invention is loaded with a viewer program for displaying the medical image data to capture specific image data from the medical image data and perform editing of the captured image data. In this case, the viewer program of the medical image data can be stored in the memory 104.

The control unit 101 executes the viewer program for the medical image data display to capture the specific image data and edit the captured image data. The control unit 101 stores the captured image data belonging to the medical image data in the memory 104. In doing so, the control unit 101 controls attribute information of the captured image data together. And, the image data captured by the viewer program and the attribute information given to the captured image data or text information corresponding to doctor's comments in a format of diagnostic recording information.

While the viewer program is executed, the workstation 100 sequentially plays back the medical image data received from the receiver 202 through the monitor. And, the workstation 100 plays back the medical image data by the frame unit.

The viewer program supports capturing of partial image data in medical image data, amendment and editing of the captured image data, attribute information of the captured image data and input of text information corresponding to doctor's comments. For operation of the viewer program, the capturing of the partial image data, the amendment and editing of the image data, the recording of the attribute information and the recording of the text information corresponding to the doctor's comments are executed. And corresponding execution results are stored in the memory 104.

The memory 104 includes a first area for storing the received medical data and the captured image data and a second area for storing the attribute information of the captured image data and the text information corresponding to the doctor's comments.

The workstation 100 provides a function for discriminating and outputting internal organs, which belong to a digestive organ from the anus to the gullet, from the medical image data received from the capsule endoscope 20.

For the internal organ discriminating and outputting function, the workstation 100 is provided with a time bar indicating a temporal length of whole image frames corresponding to the received medical image data. In this case, the time bar is displayed on the monitor.

The time bar includes a plurality of indicators. A first indicator among them indicates a current play position according to playback of the whole image frames. And, a second indicator indicates a position of an internal organ discriminating image frame among the whole image frames.

The internal organ discriminating image frame is primarily and randomly determined in the whole image frames based on the experimental values of time for the capsule endoscope 20 to pass through the internal organs belonging to the digestive organ from the gullet to the anus. The internal organ discriminating image frame is secondarily and finally determined if the user specifies one of the played image frames. For instance, if a landmark is specified on a currently played image frame by a user, the internal organ discriminating image frame can be secondarily and finally determined.

In the following description, the primarily and randomly determined internal organ discriminating image frame is named a candidate internal organ discriminating image frame and the secondarily and finally determined internal organ discriminating image frame is named a confirmed internal organ discriminating image frame.

Meanwhile, in determining a candidate internal organ discriminating image frame corresponding to a position at which an internal organ is changed from while image frames, the workstation 100 is able to make the determination based on the experimental time or the variation of a pixel value of whole image frames. And, the candidate internal organ discriminating image frame can include a plurality of image frames to compensate for a problem of accuracy.

For instance, if the capsule endoscope 20 performs capturing as many as a count of K image frames in a section at which internal organs are changed from the small intestine to the large intestine, the workstation 100 entirely determines the K image frames as candidate internal organ discriminating image frames and then marks the second indicator on points corresponding to their positions on the time bar. Preferably, an image frame corresponding to the middle of the K image frames is determined as the candidate internal organ discriminating image frame.

Preferably, the confirmed internal organ discriminating image frame is a single image frame.

The two indicators are displayed independent from each other. In particular, the first indicator is automatically shifted to an end point from a start point of the time bar according to a time flow unless there is a random manipulation. On the other hand, the second indicator is fixed thereto without shift. And, a display color or shape of the second indicator in case of the primary and random decision is represented different from that in case of the secondary and final determination.

The workstation 100 supports an internal organ discriminating mode for displaying a plurality of image frames before and after the primarily or secondarily determined internal organ discriminating image frame on the monitor simultaneously.

For instance, if the candidate or confirmed internal organ discriminating image frame represented on the time bar is selected, the workstation 100 is operable in the internal organ discriminating mode.

For instance, the workstation 100 is able to operate in the internal organ discriminating mode by a separate control button provided by an execution of the viewer program without a selection of a prescribed internal organ discriminating image frame.

In the internal organ discriminating mode, the internal organ discriminating image frame can be switched from a candidate internal organ discriminating image frame to a confirmed internal organ discriminating image frame according to a selection made by a user.

The switching of the internal organ discriminating image frame in the internal organ discriminating mode is explained as follows.

First of all, a user selects the second indicator of the time bar in the internal organ discriminating mode.

If so, the workstation 100 simultaneously displays a plurality of image frames before and after an image frame corresponding to the second indicator on the monitor.

If the user selects an image frame decided as an internal organ discriminating image from a plurality of the displayed image frames, the workstation 100 sets the selected image frame to a confirmed internal organ discriminating image frame. The workstation 100 displays the second indicator at a point of the confirmed internal organ discriminating image frame on the time bar and also displays a plurality of image frames before and after the confirmed internal organ discriminating image frame on the monitor simultaneously. Of course, a plurality of the image frames are displayed in order.

In some cases, when a plurality of the image frames before and after the candidate or confirmed internal organ discriminating image frame are simultaneously displayed on the monitor, the workstation 100 is able to enlarge and display two image frames right before and after the candidate or confirmed internal organ discriminating image frame to be distinguishable from the rest of the image frames.

For instance, if an nth image frame among whole image frames is a confirmed internal organ discriminating image frame, when (n−8)^{th} to (n+8)^{th} image frames are simultaneously displayed, (n−1)^{th}, n^{th} and (n+1)^{th} image frames are displayed on a center of a monitor by being enlarged greater than the rest of the image frames while the rest of the image frames are displayed in smaller sizes around the (n−1)^{th}, n^{th} and (n+1)^{th} image frames.

For another instance, an internal organ discriminating image frame can be displayed on the center of the monitor by being further enlarged into a largest size among the enlarged and displayed image frames.

For another instance, if a mouse pointer is placed on one of a plurality of simultaneously displayed image frames without clicking a mouse, the image frame at the corresponding position can be further enlarged and displayed as well.

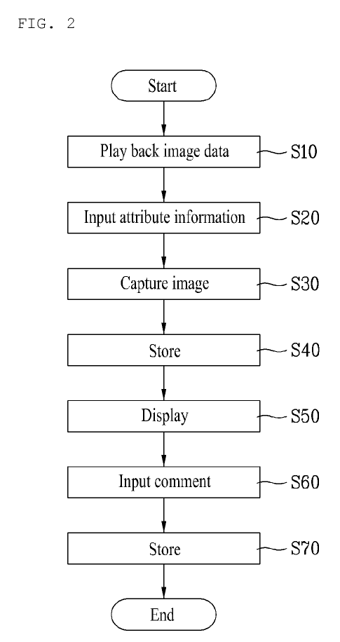

FIG. 2 is a flowchart of a process for processing medical image data according to one embodiment of the present invention.

Referring to FIG. 2, the workstation 100 reproduces medical image data received from the capsule endoscope 20 and then displays the reproduced data on the monitor [S10]. In this case, the medical image data are played back in order of capturing by a frame unit.

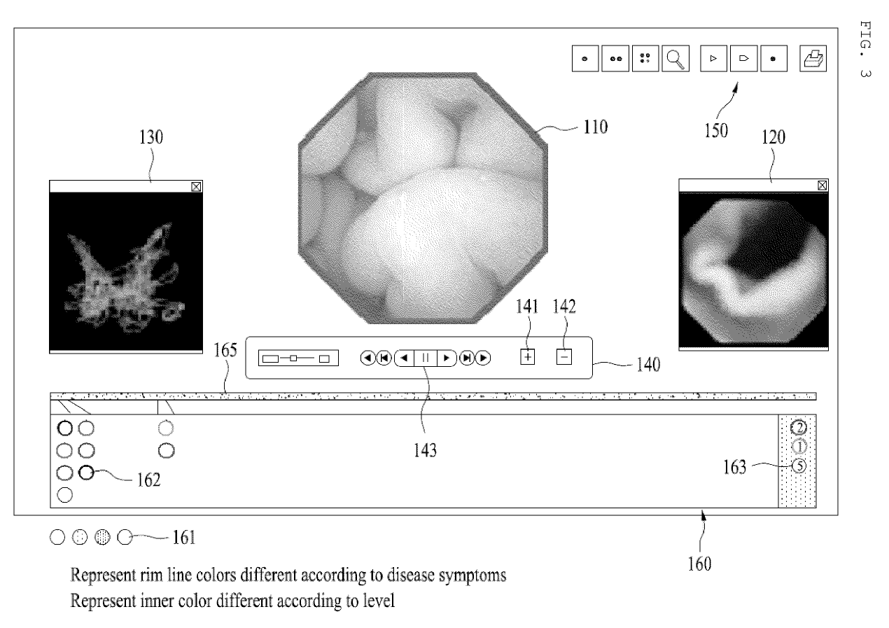

The workstation 100 executes a viewer program to enable the medical image data, as shown in FIG. 3, to be displayed on the screen of the monitor.

In particular, the medical image data, as shown in FIG. 3, are displayed within a main window 110 one by one in order. And, left and right small popup windows 120 and 130 are provided next to left and right sides of the main window 110, respectively.

In the left popup window 130, the image data currently shown in the main window 110 is displayed as a 3-dimensional image of a part of a captured digestive organ per a different internal organ position.

In the right popup window 120, a state of a digestive organ having a disease symptom is shown. For the comparison to the image displayed in the main window 110, the right popup window 120 provides a previously stored image of the same part of the image shown in the main window 110.

By the execution of the viewer program, a menu icon box 140 relevant to playback of image data is provided below the main window 110. And, the menu icon box 140 supports executions of functions of play, stop, pause 143, play speed adjust, backward, forward and landmark 142.

The landmark 142 is provided to enable a user to mark a start point of each internal organ while viewing the image data displayed in the main window 110. In particular, the landmark 142 is a means for enabling a user, i.e., a doctor to indicate an attribute for internal organ discrimination. If the landmark 142 is selected while image data is played back in the main window 110, the corresponding image data can have the attribute of landmark. A tool collection bar 150 is provided to an upper end of the monitor screen by the operation of the viewer program. The tool collection bar 150 is provided to adjust the number and sizes of images of the image data played back in the main window 110.

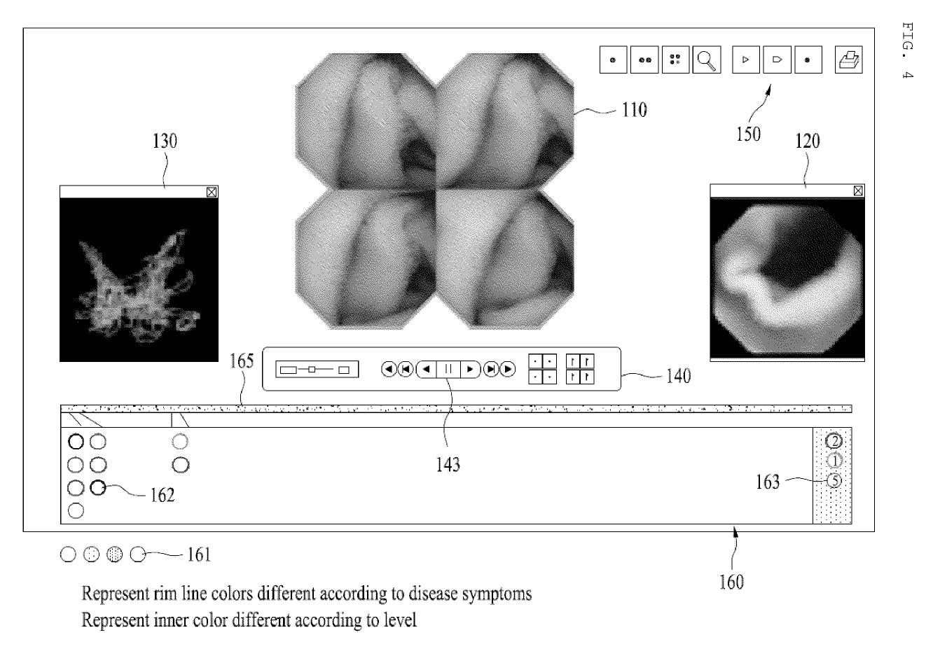

Besides, the main window 100, as shown in FIG. 4, can be divided into a plurality of screens.

For instance, the tool collection bar 150 is provided with a manipulation button for dividing the screen of the main window 100 into two or four equal parts. If the manipulation button is selected, several images are displayed on the two or four equally-divided screens of the main window 110. Of course, a plurality of the images displayed on the two or four equally-divided screens are sequentially arranged according to order of capturing time. In particular, a plurality of image data captured in a predetermined time section among whole image data received from the capsule endoscope 20 are simultaneously displayed and they are simultaneously displayed on the tow or four equally-divided screens. Alternatively, a plurality of image data received for a predetermined time section among whole image data are simultaneously displayed on the divided screens.

An interpretation window 160 is provided to a lower end of the screen by the execution of the viewer program. In the interpretation window 160, icons of the captured image data are collected. The icon is a software image tool for representing a portion of attribute information of the captured image data as a colored image shape and executing a command for calling the corresponding image data according to a selection of the image shape.

An additional icon 163 is provided to a right side within the interpretation window 160 to represent the number of icons indicating the same attribute among the icons collected within the interpretation window 160.

The additional icon 163 represents the attribute as a colored image shape in the same manner of the generally collected icon. So, if a digit is represented within a rim of the additional icon 163, it indicates how many icons having the same attribute of the colored image shape are created. If an icon indicating the same attribute is further created, the digit within the rim of the corresponding additional icon is incremented as many as the number of the created icons.

Meanwhile, the time bar 165 is provided above the interpretation window 160.

The time bar 165 indicates a temporal length of while image frames corresponding to the received medical image data and also indicates that the captured image data of the icon provided within the interpretation window 160 is located at a specific point of the temporal length of the whole image frames.

As mentioned in the foregoing description, when the whole image frames are played back, the time bar 165 uses the first indicator to indicate that the image data played back in the main window 110 is located at a specific point of the temporal length of the whole image frames and also uses the second indicator to indicate a position of the internal organ discriminating image frame among the whole image frames. In this case, the second indicator is a software image tool for indicating that specific image data is an image for discriminating an internal organ using a colored image and executing a command for calling the corresponding image data according to a selection of the image shape.

Subsequently, attribute information input is then carried out while the medical image data is being played back [S20].

The process for the attribute information input is explained in detail as follows.

First of all, if the user selects the pause 143 on the playback screen of the image data, the currently played image data is displayed as a still picture in the main window 110 under the control of the control unit 101 of the workstation 100.

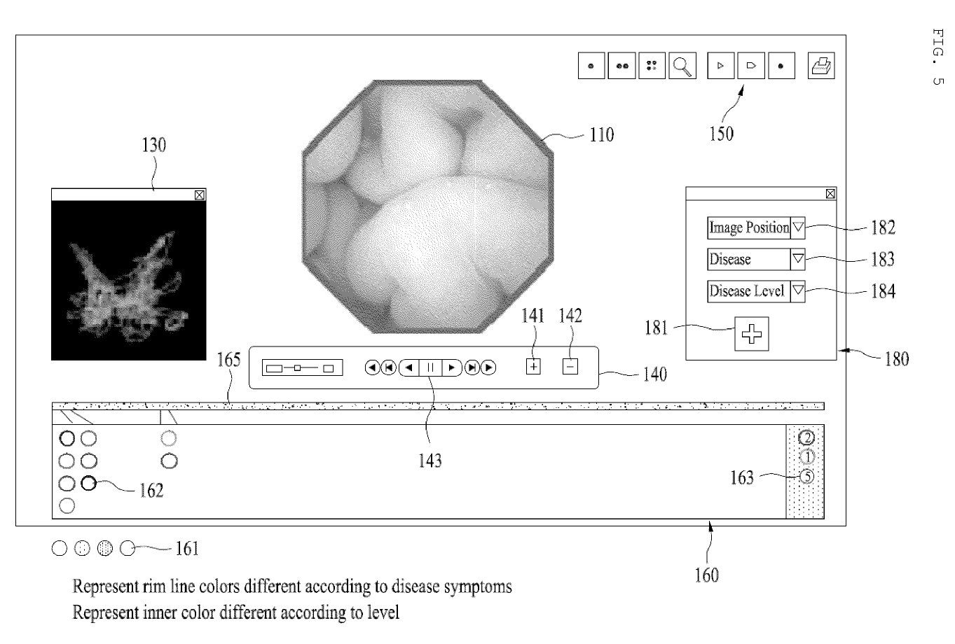

If the user selects the button 141 for the attribute information input within the menu icon box 140, a popup window 180, as shown in FIG. 5, for the attribute information input is shown by the execution of the viewer program. In this case, the user is able to input attribute information on the image data that is currently played back as the still picture in the main window 110 via the popup window 180.

The attribute information is a result of the still picture diagnosed by the user and contains disease information indicating that the played image matches a predetermined disease and disease seriousness information indicating a level of the disease if the played image matches the predetermined disease.

The attribute information on the captured image data includes the aforesaid various diagnostic results, a date and time at which the corresponding image data is captured by the capsule endoscope, and the aforesaid landmark information.

To enable various items of the attribute information to be selected, the popup window 180 for the attribute information input includes an internal organ selecting button 182 for enabling one of various internal organs of the digestive organ to be selected, a disease selecting button 183 for enabling a type of disease indicated by a played image to be selected, and a disease seriousness level selecting button 184 for enabling a seriousness level of disease indicated by the played image to be selected.

In case that the internal organ selecting button 182 is selected, a list containing per internal organ positions is provided to select a position of one of various internal organs including an entry of the gullet, a middle part of the large intestine, a latter part of the small intestine and the like.

In case that the disease selecting button 183 is selected, a list containing disease types is provided to select a disease state of tumor, bleeding and the like at the internal organ position selected by the internal organ selecting button 182.

In case that the disease seriousness level selecting button 184 is selected, a level list indicating a seriousness level of disease is provided to select the disease seriousness level indicating a level of the state of the corresponding disease selected by the disease selecting button 183.

After three kinds of the attribute informations of the image data currently played back as the still picture in the main window 110 have been selected via the three buttons 182, 183 and 184, if a capture button 191 provided to the popup window 180 for the attribute information input is selected, a current still picture of the main window 110 is captured [S30].

As the still picture is captured, the control unit 101 interconnects the played image data with the selected attribute information and then stores it in the memory 104 [S40]. In this case, the played image data is stored as a photo file. And, the photo file has various attribute informations containing the above-selected three kinds of attributes.

Thus, as the image data is stored in the memory as the photo file having the attribute information, an icon of the captured image data is created in the interpretation window 160. In this case, the icon presents a portion of the attribute information of the photo file and is able to indicate information corresponding to the aforesaid various diagnostic results. It is able to then read the stored photo file through a selection of the icon.

For instance, the icon 162 is a circular icon and represents attribute information differently using a color of a circular rim, a color within the circular rim, and a thickness of the color within the circular rim.

The circular shape of the icon 162 is taken as an example. And, more variations of the shape of the icon 62 are available.

For instance, a circular rim color indicates a type of disease and a level of seriousness of disease is represented as a color thickness within the rim. The aforesaid additional icon 163 is implemented with the same shape of the icon 162. Yet, digit for indicating how many icons having the same attribute are created among icons indicating the attribute of the image data is further represented within the circular rim of the additional icon 163.

The icon 162 is connected to one point or one section of the time bar 165 through a connecting line. So, it indicates that the corresponding image data interconnected to the icon 162 is captured at which timing point of the total time taken for the capturing by the capsule endoscope.

Time attribute is displayed in a manner that the icon 162 is connected to the one point or section of the time bar 165 by the connecting line. As the captured image data is stored as the photo file by being interconnected to various kinds of attribute information, it is assumed that the icon 162 is created in the interpretation window 160.

If the created icon 162 is selected by the user 162, the control unit 101 reads the photo file interconnected to the selected icon 162 from the memory 104. The control unit 101 then displays the corresponding photo file [S50].

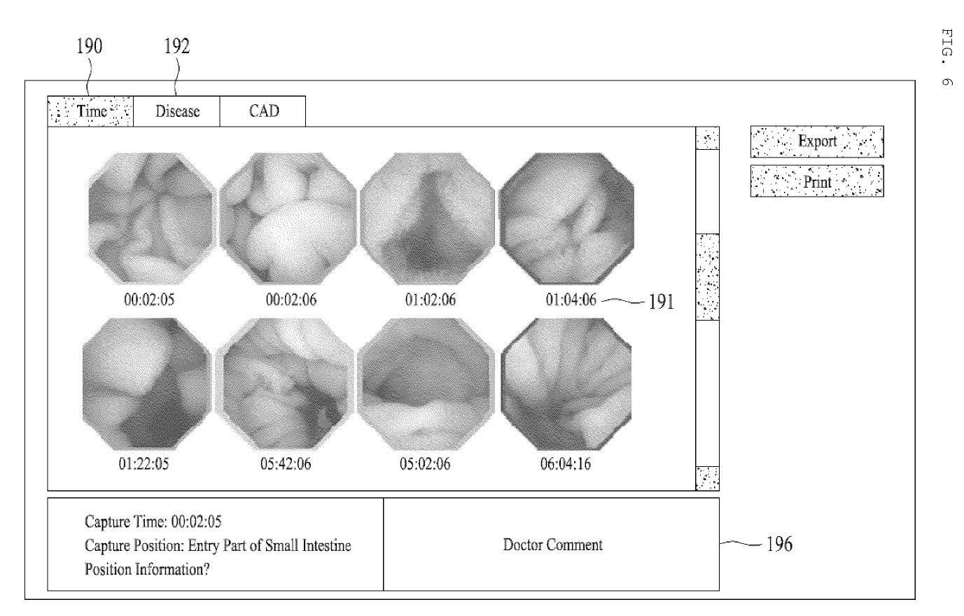

FIGS. 6 to 8 show various screen configurations for displaying stored photo files.

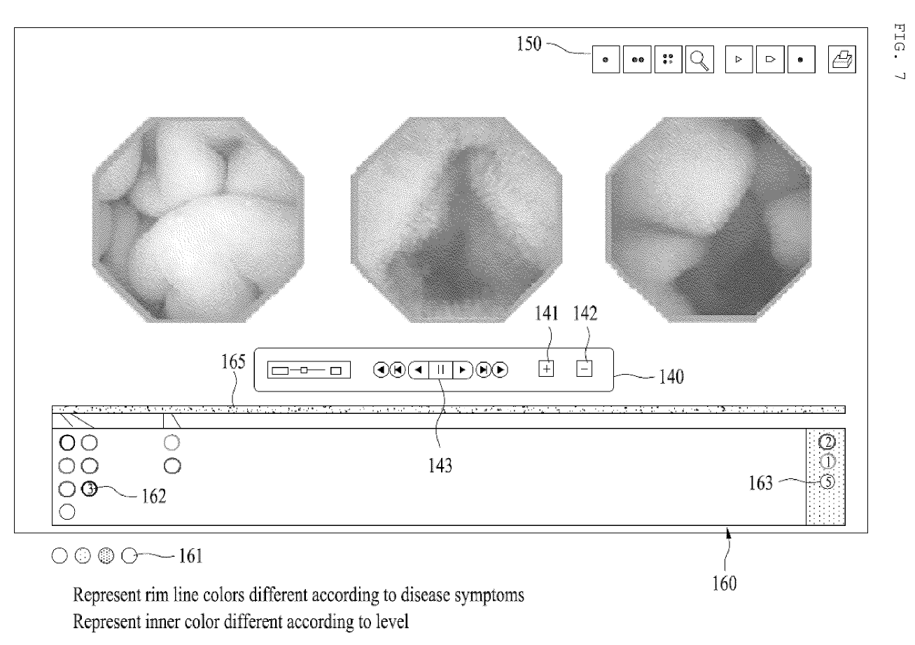

Referring to FIG. 7, it is able to display the interpretation window 160 on the screen for displaying stored photo files as well.

If a specific icon collected in the interpretation window 160 is selected, the control unit 101 reads the photo file interconnected to the selected icon from the memory 104 and then displays it in a separate window 90. The separate window 90 provides a tool for editing an image of the read photo file. N the following description, the separate window 90 shall be named an editing window.

If one of the additional icons having digits represented within their rims is selected from the interpretation window 160, the control unit 101 reads photo files having the same attribute of the selected additional icon from the memory 104 and then displays the read photo files in a manner of sorting them in a plurality of windows, respectively.

If the additional icon having a digit 3 represented within its rim, as shown in FIG. 7 for example, is selected, the control unit 101 reads three photo files having the same attribute of the selected additional icon from the memory 104 and then displays the three photo files in a manner of sorting them in three windows, respectively. The sorting order of the displayed photo files is based on time.

Yet, in case that it is unable to display all photo files on one screen since digits within the time of the additional icon are too big, a scroll bar is further formed to support a down/up scroll of the screen.

The viewer program of the present invention provides menus 190 and 192 for changing the sorting scheme in displaying a plurality of photo files having the same attribute.

If ‘time’ which is a menu for time based sorting is selected, a photo file having an earliest capturing time, as shown in FIG. 6, is preferentially sorted based on an attribute of the photo file. And, a capturing time 191 is displayed on a played image of each photo file. In this case, rims of windows for displaying images of photo files differ from each other in color according to attribute informations of the photo files, respectively.

Preferably, a rim color of the window is determined based on color and thickness of an icon interconnected to the corresponding photo file. For instance, a color of the same color series is used to indicate the same disease but thickness of the color differs to indicate a seriousness level of the corresponding disease.

In sorting and displaying a plurality of photo files, a character color of the capturing time 191 displayed below each of the windows for displaying images of the photo files differs according to a landmark attribute of each of the photo files, i.e., an attribute for internal organ discrimination.

In particular, in order to discriminate different internal organs from each other, if images of photo files correspond to internal organs differing from each other, respectively, display colors of the capturing times 191 respectively displayed below the corresponding windows differ from each other.

Thus, in case that a plurality of photo files are sorted with reference to time, display types of rims and capturing times of windows for displaying images of the photo files are implemented different according to attributes of the photo files, respectively.

Hence, when images of a plurality of photo files are displayed, a user is able to recognize an image of a specific internal organ, a specific disease symptom of the internal organ or a seriousness level of the disease symptom at a glance through colors and thickness of rims of windows for the displayed images and display colors of capturing times.

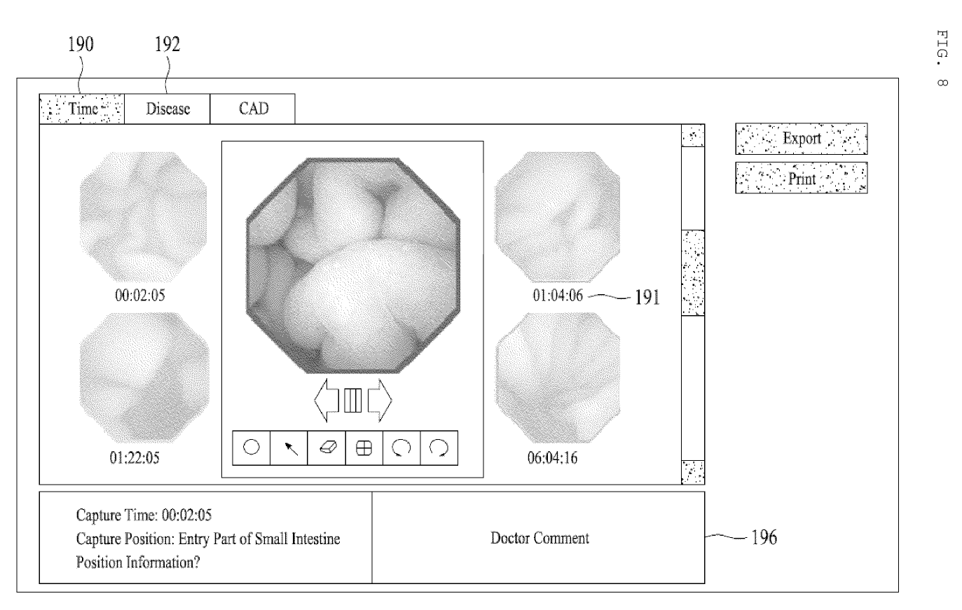

In the viewer program of the present invention, when a plurality of photo files having the same attribute are displayed, if an image is selected from the photo files respectively displayed in windows, the control unit 101, as shown in FIG. 8, displays the selected image in the editing window 90 for the image editing.

The editing window 90 shown in FIG. 8 is provided with an arrow 91 for the switching to an image in before & after sorting order of an image selected from sorted images.

The viewer program of the present invention provides a command input window 196 for inputting user's comment information on an image displayed in the editing window 90.

If comment information on the image currently displayed in the editing window 90 is inputted to the command input window 196, the control unit 101 stores the inputted comment information in a manner of interconnecting the inputted comment information to a photo file of the image currently displayed in the editing window 90 [S70].

For another instance, as a specific icon collected in the interpretation window 160 is selected, if an image of a photo file interconnected to the selected icon is displayed in the editing window 90, the viewer program of the present invention is able to provide the command input window 196 for inputting user's comment information on the image displayed in the editing window 90. If the user inputs the comment information on the image currently displayed in the editing window 90 is inputted to the command input window 196 [S60], the control unit 101 stores the inputted comment information in a manner of interconnecting the inputted comment information to a photo file of the image currently displayed in the editing window 90.

Yet, if the image currently displayed in the editing window 90 is to be edited, the inputted comment information is stored in a manner of interconnecting the inputted comment information to an edited photo file.

If the command input window 196 is activated, a cursor is generated within the command input window 196. If a text is inputted to the command input window 196, the corresponding text is stored as comment information in a manner of interconnecting the corresponding comment information to a photo file of an image currently displayed in the editing window 90. In this case, the editing window 90 is provided with an editing bar for putting a specific mark on the image currently displayed in the editing window 90, rotating the image, deleting the image in part, and the like. When the editing window 90 is activated, the user is able to mark a bleeding part or a part having a polypus on an image using the editing bar, rotate the corresponding image or delete the image in part.

Thus, if the comment information input and the image editing for the image currently displayed in the editing window 90 are completed, the inputted comment information is stored by having the inputted comment information interconnected to the edited photo file [S70]. And, the old photo file before the editing is automatically deleted or can be separately stored as a backup file.

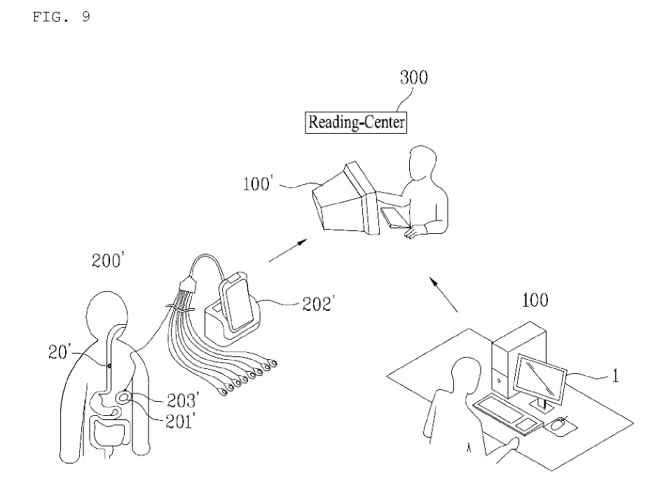

FIG. 9 is a diagram for a configuration of a capsule endoscope system according to another embodiment of the present invention.

Since configurations and operations of a capsule endoscope 20′, a sensor 201′, a receiver 202′, a sensor pad 203′ and a workstation 100′ in the configuration shown in FIG. 9 are similar to those explained in the descriptions of FIGS. 1 to 8, details thereof shall be omitted in the following description. Yet, the workstation 100′ shown in FIG. 9 is established in a reading center 300.

The workstation 100′ of the reading center 300 receives medical image data stored in the receiver 202′ from the receiver 202′ and then forwards it to a user workstation 100.

Preferably, the workstation 100′ of the reading center 300 receives the medical image data stored in the receiver 202′ and then generates a capture image for the received medical image data. Subsequently, the workstation 100′ of the reading center 300 transmits the generated capture image to the user workstation 100.

The user workstation 100 is loaded with comment information on the received capture image and a viewer program for jobs of image editing and the like. The viewer program is identical to that explained in FIGS. 1 to 8.

The workstation 100′ of the reading center 300 processes the medical image data received from the capsule endoscope 20′ and then transmits the processed data to the user workstation 100. For this, the workstation 100′ of the reading center 300 includes an input unit, an output unit, a control unit, and a memory.

The workstation 100′ of the reading center 300 and the user workstation 100 are mutually connected via a wire/wireless network such as Internet for transmission and reception of the medical image data.

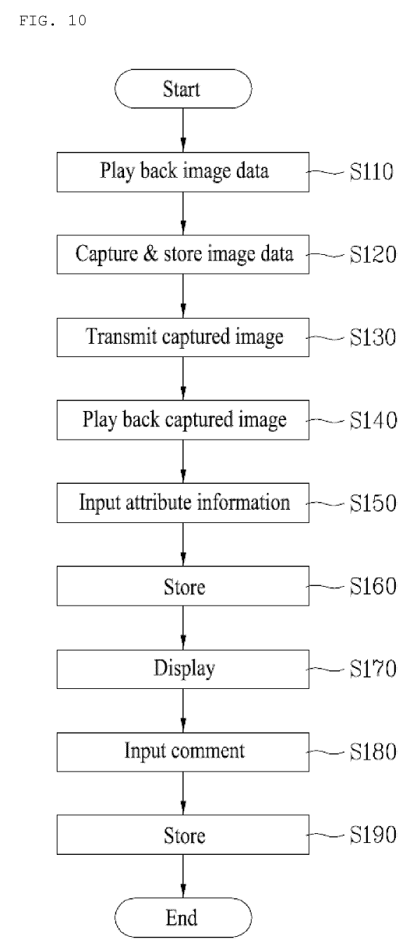

FIG. 10 is a flowchart of a process for processing medical image data according to another embodiment of the present invention, in which image data processing procedures including a procedure for providing a captured image based on the system shown in FIG. 9 are shown.

Referring to FIG. 10, the workstation 100′ of the reading center 300 reproduces the medical image data received from the capsule endoscope 20 and then displays the reproduced data via a monitor [S110]. In this case, the medical image data are reproduced by a frame unit in order to being captured.

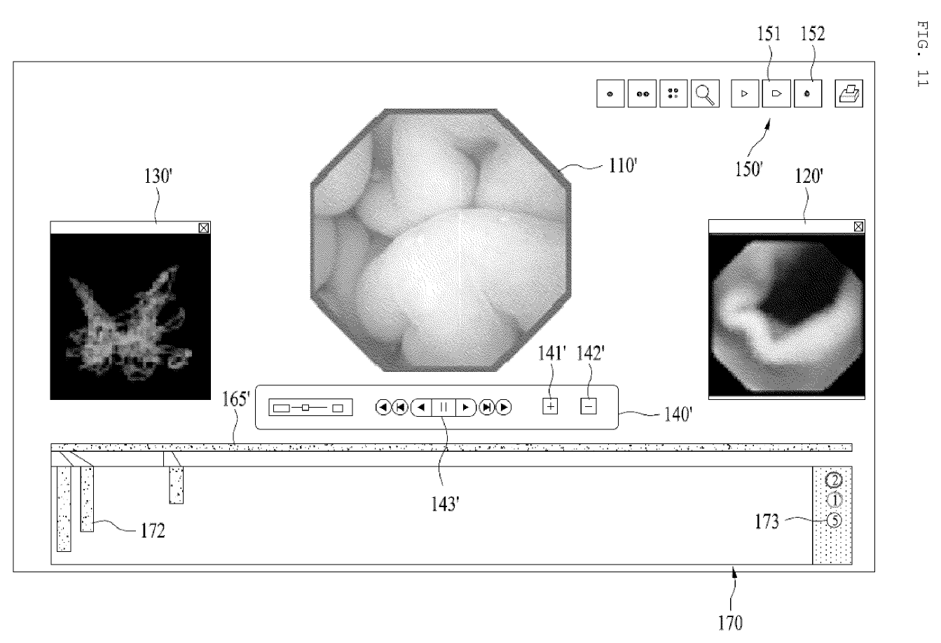

The workstation 100′ of the reading center 300 is provided with a viewer program for displaying the medical image data in a format shown in FIG. 11 on a monitor screen.

The workstation 100′ of the reading center 300, as shown in FIG. 11, displays the medical image data one by one within a main window 110′ through execution of the viewer program.

Small-size popup windows 120′ and 130′ are provided next to left and right sides of the main window 110′, respectively.

The main window 110′ and the popup windows 120′ and 130′ next to the left and right sides of the main window 110′ are operative in the same manner of the main windows 110 and the left and right popup windows 120 and 130 explained in the description of FIG. 3.

The identical operative implementations of the main window 110′ and the popup windows 120′ and 130′ are summarized in brief as follows. First of all, functions of play, stop, pause 143, play speed adjust, backward, forward and landmark 142 for the image data are performed through the main window 110′. Secondly, a count and sizes of screens for the image data played in the main window 110′ are adjusted. Thirdly, a screen of the main window 110′ is divided into a plurality of parts and a plurality of the parts are then sorted. For these operations, the workstation 100′ of the reading center 300 provides various tools for adjusting rotations, divisions and sizes of the displayed images through the execution of the viewer program.

Preferably, the viewer program of the workstation 100′ does not provide a function for managing an attribute information input of the image data played back in the main window 110′.

Alternatively, the viewer program loaded in the workstation 100′ of the reading center 300 can be implemented to reproduce the medical image data through the main window 110′ only.

When an image is displayed on the monitor screen as the image data is reproduced through the execution of the viewer program, the control unit of the workstation 100′ captures a current image in the main window 110′ according to a selection command given by an endoscope surveillant of the reading center 300 and then stores the captured image as a photo file [S120].

The image capture and storage are executed if the endoscope surveillant inputs a command for capturing images of a disease symptom possible part to the workstation 100 while a plurality of images are displayed via the main window 110′. In this case, the control unit of the workstation 100′ stores the captured image only as a photo file without inputting attribute information.

A time bar 165′ is provided to the screen through the execution of the viewer program.

The time bar 165′ indicates a temporal length of whole image frames corresponding to the received medical image data. Through the execution of the viewer program, a window 170 for representing a cumulative count of images captured per a view as a bar type 172 or a time information list is further provided below the time bar 165′.

By the execution of the viewer program, a tool collection bar 150′ is provided to an upper end of the monitor screen. The tool collection bar 150′ is to adjust a screen count and size of image data played back in the main window 110′ and capture a portion of the image data played back in the main window 110′.

The tool collection bar 150′ includes an extraction button 151 for extracting and capturing a portion (disease symptom part) of the image data played back in the main window 110 and a blood stain search button 152 for capturing a blood stain part by searching the played image data for the blood stain part.

The workstation 100′ of the present invention supports a software image analysis function for automatically capturing disease symptom possible image data from the received medical image data or a portion or a blood stain part of the image data.

In particular, through the image analysis function, a diagnose-impossible image data is extracted from whole image frames corresponding to the medial image data. For this, the image analysis function removes unnecessary image frames by analyzing a color and pixel value per an image frame while the medical image data are played back.

For instance, if a considerable amount of leftovers exist in an internal organ, medical examination is impossible through playback images despite capturing the images. In this case, the corresponding image frames are automatically extracted and removed through the image analysis function. Occasionally, the workstation 100′ of the present invention is able to automatically capture and store analyzable image frames except the unnecessary image frames.

Likewise, if the blood stain search button 152 is selected, a color (red) and pixel value indicating a blood stain are analyzed for each played image data through the image analysis function and an image frame having the blood stain exist therein is then automatically captured.

The image analysis function can be primarily applied to detection of an internal organ discriminating image frame. In particular, the image analysis function can be used in automatically detecting the above-mentioned internal organ discriminating image frame.

The image data captured from the whole medical image data is stored in the memory of the workstation 100′.

Subsequently, if the user workstation 100 makes a request for the captured image data through a network, the workstation 100′ of the reading center 300 transmits the image data stored in the memory to the user workstation 100 in response to the request [S130].

The user workstation 100 having received the captured image data from the workstation 100′ of the reading center 300 executes the viewer program loaded therein and then plays and displays the received image data trough the monitor according to the execution of the viewer program [S140].

Subsequently, an attribute information input is carried out while the image data is being played back [S150].

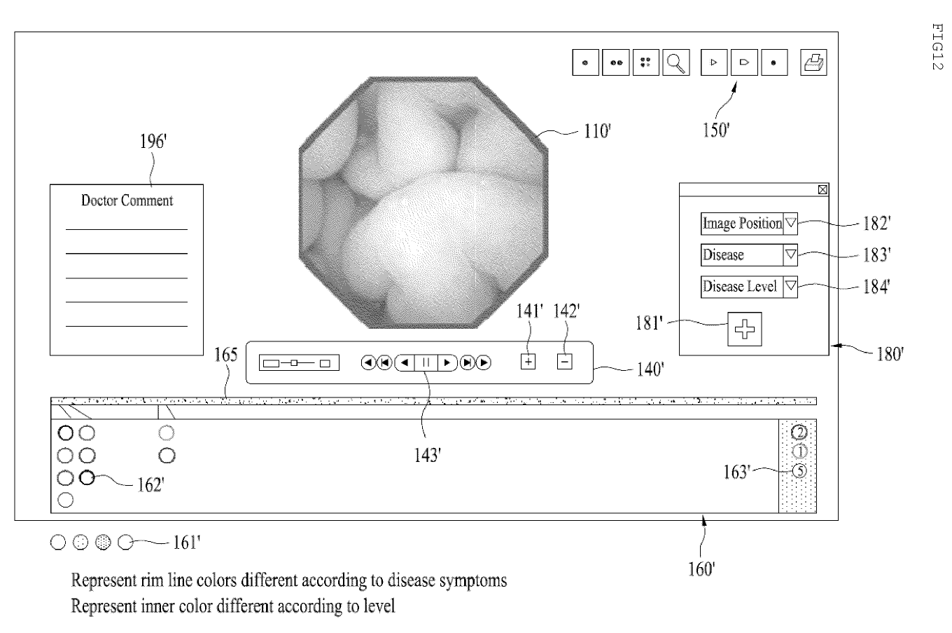

The progress of the attribute information input is explained in detail with reference to FIG. 12 as follows.

First of all, if a user selects an attribute information input button 141′ for an attribute information input within a menu icon box 140′ while image data is currently played back, an image currently played and displayed on the screen stops under the control of the control unit of the workstation 100′.

Subsequently, a popup window 180′ for the attribute information input appears by the execution of the viewer program. A user is then able to input attribute information on the image data currently played as a still picture on the main window 110′ via the popup window 180′.

The input of the attribute information is carried out in the same manner explained in the descriptions of FIGS. 1 to 8. In particular, as the attribute information, which is the diagnostic result by the user and contains position information indicating that a played image corresponds to a specific position of a prescribed internal organ, disease information indicating that the played image matches a predetermined disease and disease seriousness information indicating a seriousness level of the disease if the played image matches the predetermined disease, is selected by the user via the popup window 180′, attribute information according to the selected attribute information is inputted.

To enable various items of the attribute information to be selected, the popup window 180′ for the attribute information input includes an internal organ selecting button 182′ for enabling one of various internal organs of the digestive organ to be selected, a disease selecting button 183′ for enabling a type of disease indicated by a played image to be selected, and a disease seriousness level selecting button 184′ for enabling a seriousness level of disease indicated by the played image to be selected.

In case that the internal organ selecting button 182′ is selected, a list containing per internal organ positions is provided to select a position of one of various internal organs including an entry of the gullet, a middle part of the large intestine, a latter part of the small intestine and the like.

In case that the disease selecting button 183′ is selected, a list containing disease types is provided to select a disease state of tumor, bleeding and the like at the internal organ position selected by the internal organ selecting button 182′.

In case that the disease seriousness level selecting button 184′ is selected, a level list indicating a seriousness level of disease is provided to select the disease seriousness level indicating a level of the state of the corresponding disease selected by the disease selecting button 183′.

After three kinds of the attribute informations of the image data currently played back as the still picture in the main window 110′ have been selected via the three buttons 182′, 183′ and 184′, if a capture button 181′ provided to the popup window 180′ for the attribute information input is selected, the control unit interconnects the played image data to the selected attribute information and then stores it in the memory [S160].

In this case, the played image data is stored as a photo file. And, the photo file has various attribute informations containing the above-selected three kinds of attributes.

Thus, as the image data is stored in the memory as the photo file having the attribute information, an icon of the corresponding image data is created in the interpretation window 160′. It is able to then read the stored photo file through a selection of the icon.

Thus, as the photo files having the attribute information are stored one by one, icons corresponding to the stored photo files are created and then collected in the interpretation window 160′.

The icon collected in the interpretation window 160′ indicates a portion of the attribute information of the photo file and represents information corresponding to various diagnostic results as a color and image shape. So, the user is able to confirm the diagnostic result of the image corresponding to the icon from the color and image shape of the icon in the interpretation window 160′. The color or image shape refers to that of the aforesaid example.

The attribute information on the stored photo file includes various diagnostic results represented by the icon, a date and time at which the corresponding image data is captured by the capsule endoscope 20′, and the aforesaid landmark information. Of course, the photo file having the landmark information as the attribute information corresponds to the aforesaid internal organ discriminating image frame.

In order to indicate an attribute of the time for capturing the image data by the capsule endoscope 20′, the icon is connected to one point or one section of the time bar 165 through a connecting line. So, it indicates that the corresponding image data interconnected to the icon is captured at which timing point of the total time taken for the capturing by the capsule endoscope 20′.

An additional icon 163′ is provided to a right side within the interpretation window 160′ to represent the number of icons indicating the same attribute among the icons collected within the interpretation window 160′. The additional icon 163′ has been explained in the foregoing description, of which details shall be omitted in the following description.

If the user selects the created icon, the control unit reads the photo file interconnected to the icon from the memory. The control unit then displays the photo file [S170].

While the read photo file is displayed, the viewer program of the present invention provides a comment input window 196′ for user's comment information input.

After the comment input window 196′ has been activated, if comment information on the displayed photo file is inputted as a text to the comment input window 196′ [S180], the control unit interconnects the inputted comment information to the displayed photo file and then stores it [S190].

The progress sequences of the attribute information input and the user's comment information input in FIGS. 2 to 10 may vary according to how the present invention is implemented. In particular, the comment information input may be carried out ahead of the attribute information input. The case of inputting the attribute information after completion of the comment information input can be understood from the aforesaid description, of which details shall be omitted in the following description.

Explained in the following description is an internal organ discriminating mode for simultaneously displaying a plurality of frames before and after a primarily or secondarily determined internal organ discriminating frame on a monitor.

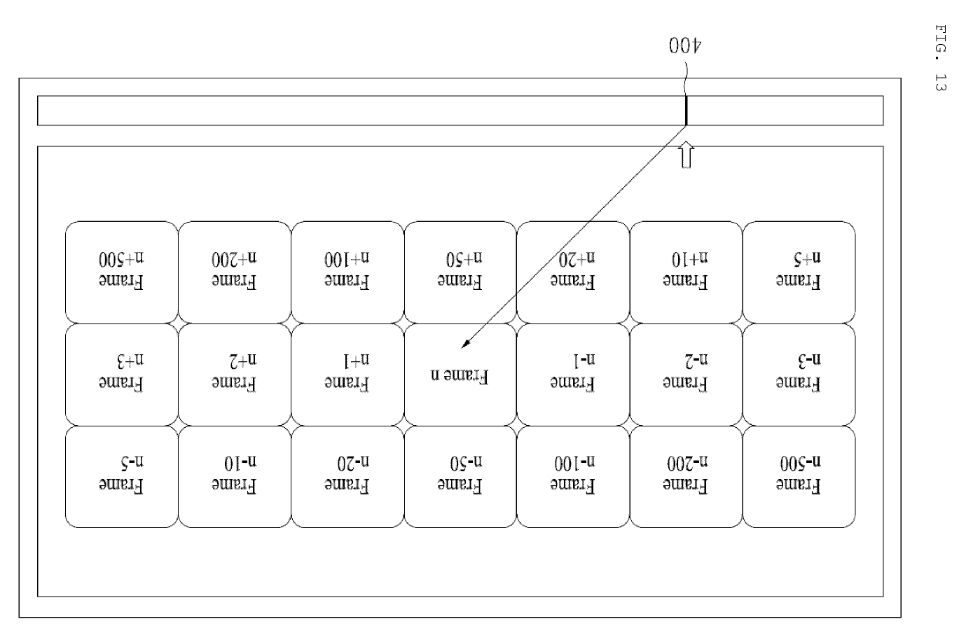

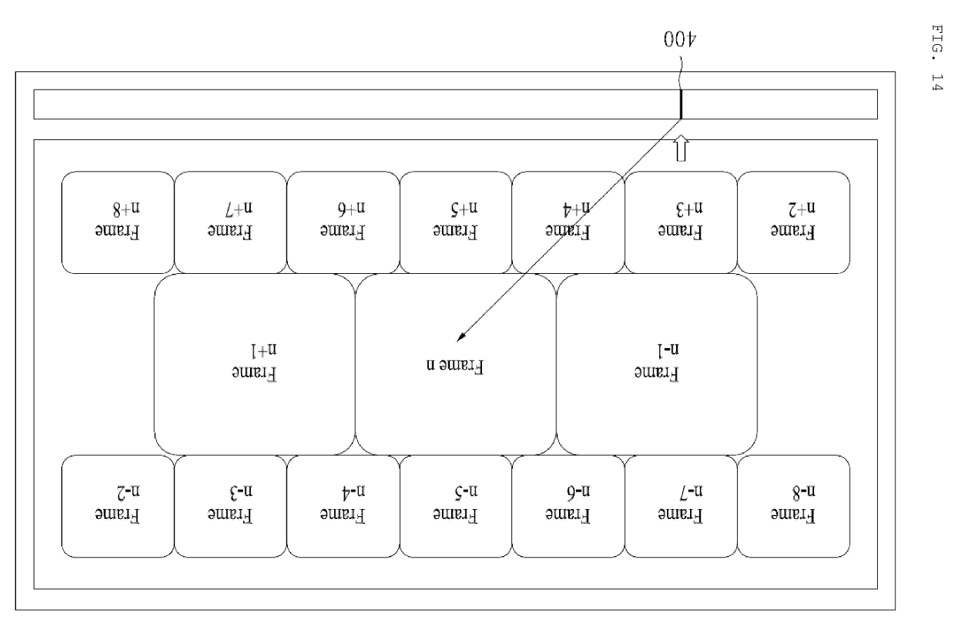

FIG. 13 and FIG. 14 are diagrams for played images in internal organ discriminating mode according to various embodiments of the present invention. FIG. 13 shows an example that a plurality of image frames are sorted in the same size, while FIG. 14 shows an example that image frames right before and after an internal organ discriminating image frame are enlarged and displayed distinguishable from the rest of the image frames.

In case of entering an internal organ discriminating mode for outputting images of internal organs belonging to the digestive organ from the gullet to the anus from the medical image data received from the capsule endoscope 20, a plurality of image frames before and after a primarily or secondarily determined internal organ discriminating frame are simultaneously displayed on the monitor according to the execution of the viewer program of the workstation 100.

Second indicator 400 indicating a position of an internal organ discriminating image frame is displayed on a time bar indicating a temporal length o whole image frames.

The second indicator 400 indicates a view of a candidate internal organ discriminating image from determined primarily and randomly. And, its position may be changed on the time bar if a user specifies a landmark attribute in an image frame that is played later.

FIG. 13 shows an example that a plurality of image frames frame n−500 before an internal organ discriminating image frame frame n corresponding to the second indicator 400 and a plurality of image frames n+500 after the internal organ discriminating image frame frame n are simultaneously displayed centering on the internal organ discriminating image frame frame n, while whole image frames are selected and displayed in part.

In an internal organ discriminating mode of the present invention, a plurality of image frames are displayed in a matrix form. In particular, FIG. 13 shows an example that the image frames are displayed in 3×7 matrix form.

Referring to FIG. 13, an internal organ discriminating frame is displayed at a central matrix position (2, 4). Former image frames are displayed by one timing toward a left side and latter image frames are displayed by one timing toward a right side, centering on the internal organ discriminating frame on the second row as a middle row.

On the first row as an upper row above the middle row, i.e., the second row, selected image frames are displayed by incrementing an interval of the timing. On the third row as a lower row below the middle row, i.e., the second row, selected image frames are displayed by incrementing an interval of the timing.

In particular, on the first row, as shown in FIG. 13, image frames at views, of which inter-image frame timing intervals are respectively decremented by 5, 10, 30, 50, 100 and 300 frames, are sequentially displayed toward the left side from the right side. On the third, as shown in FIG. 13, image frames at views, of which inter-image frame timing intervals are respectively incremented by 5, 10, 30, 50, 100 and 300 frames, are sequentially displayed toward the right side from the left side. Therefore, ‘frame n−5’, ‘frame n−10’, ‘frame n−20’, ‘frame n−50’, ‘frame n−100’, ‘frame n−200’ and ‘frame n−500’ are sequentially arranged on the first row. On the second row, ‘frame n−1’, ‘frame n−2’ and ‘frame n−3’ are sequentially arranged to the left side of the internal organ discriminating image frame frame n. On the second row, ‘frame n+1’, ‘frame n+2’ and ‘frame n+3’ are sequentially arranged to the right side of the internal organ discriminating image frame frame n. On the third row, ‘frame n+5’, ‘frame n+10’, ‘frame n+20’, ‘frame n+50’, ‘frame n+100’, ‘frame n+200’ and ‘frame n+500’ are sequentially arranged toward the right side from the left side.

As the user specifies the landmark attribute in the currently played image frame, if the internal organ discriminating image frame is changed, i.e., if a candidate internal organ discriminating image frame is changed into a confirmed internal organ discriminating image frame, a position of the second indicator 400 is changed on the time bar as well. Simultaneously, a display color of the second indicator 400 may be changed.

FIG. 14 shows an example that image frames are displayed in a similar matrix form. In simultaneously displaying a plurality of image frames before and after an internal organ discriminating image frame frame n, the internal organ discriminating image frame frame n and two image frames right after and before the internal organ discriminating image frame frame n are enlarged and displayed distinguishable from the rest of the image frames.

Referring to FIG. 14, an internal organ discriminating image frame frame n is displayed in an enlarged size at a central position (2, 4) of the matrix. On a second row as a middle row, a right former image frame frame n−1 is displayed in an enlarged size left to the internal organ discriminating image frame frame n and a right latter image frame frame n+1 is displayed in an enlarged size right to the internal organ discriminating image frame frame n.

In FIG. 14, unlike FIG. 13, a selecting range of image frames to be displayed is not made to include whole image frames but made to include ‘frame n−8’ to ‘frame n+8’ corresponding to portions of the whole frames.

On a first row shown in FIG. 14, image frames at views, of which inter-image frame timing intervals are decremented by 1 frame toward the left side from the right side, are sequentially displayed. On a third row, image frames at views, of which inter-image frame timing intervals are incremented by 1 frame toward the right side from the left side, are sequentially displayed. Hence, ‘frame n−2’, ‘frame n−3’, ‘frame n−4’, ‘frame n−5’, ‘frame n−6’, ‘frame n−7’ and ‘frame n−8’ are sequentially arranged from the right side to the left side on the first row. And, ‘frame n+2’, ‘frame n+3’, ‘frame n+4’, ‘frame n+5’, ‘frame n+6’, ‘frame n+7’ and ‘frame n+8’ are sequentially arranged from the left side to the right side on the third row.

Accordingly, in the present invention, medical image data received from a capsule endoscope is stored by being interconnected to various kinds of attribute information. Such software tools for indicating various kinds of summarized attribute information as icons, time bar and the like are provided to a screen, whereby interconnected image data are read and displayed via the tools.

And, the present invention provides software tools for displaying image data in various forms to a screen, thereby processing (editing, dividing, sorting, etc.) displayed images using the tools.

It will be apparent to those skilled in the art that various modifications and variations can be made in the present invention without departing from the spirit or scope of the inventions.

Thus, it is intended that the present invention covers the modifications and variations of this invention provided they come within the scope of the appended claims and their equivalents.

What is claimed is:

1. A method of processing image data, which is received from a capsule endoscope by a workstation, comprising the steps of:
playing back the received image data on a screen;
if a diagnostic result from a played image of the image data is inputted, storing a file corresponding to the mage data by interconnecting the file to the inputted diagnostic result, wherein the diagnostic result is stored as attribute information of the file;
creating an image tool indicating the attribute information;
displaying the created image tool on the screen;
if the displayed image tool is selected, reading the stored file and the stored attribute information; and
displaying the read file and the read attribute information on the screen.

2. The method of claim 1, further comprising the steps of:
displaying a time bar indicating a temporal length of whole image data received from the capsule endoscope; and
displaying the image tool by connecting the image tool to one point or one section of the time bar to display a time attribute of the stored file.

3. The method of claim 2, wherein if the stored file corresponds to the image data for discriminating different internal organs of a digestive organ from each other, an indicator for an internal organ discrimination is displayed at a view corresponding to the time attribute of the stored file on the time bar.

4. The method of claim 3, wherein if the displayed indicator is selected, a plurality of image frames before an image frame (frame n) of the file and a plurality of image frames after the image frame (frame n) among whole image frames of the image data including the image frame (frame n) of the file are simultaneously displayed on the screen.

5. The method of claim 4, wherein in displaying a plurality of the image frames, a plurality of the image frames before the image frame (frame n) and a plurality of the image frames after the image frame (frame n) are arranged in order of timing.

6. The method of claim 4, wherein in displaying a plurality of the image frames, the image frame (frame n) is displayed by being enlarged larger.

7. The method of claim 4, wherein the image frame (frame n), the image frame (frame n−1) right before the image frame (frame n) and the image frame (frame n+1) right after the image frame (frame n) are displayed by being enlarged lager.

8. The method of claim 1, further comprising the step of displaying an additional image tool indicating a count of the image tool and other image tools indicating the same attribute information of the image tool.

9. The method of claim 1, wherein in playing back the received image data, the received image data are sequentially arranged on divided screens according to a time flow together with other image data received from the capsule endoscope.

10. The method of claim 1, wherein if a start point mark of an internal organ is inputted from the played image of the image data, an attribute for discriminating different internal organs of a digestive organ is further included in the attribute information and then stored.

11. The method of claim 10, wherein if the attribute for the different internal organ discrimination is further included in the stored file, an indicator of the file is marked on a view corresponding to a time attribute of the stored file on a time bar indicating a temporal length of the whole image data received from the capsule endoscope.

12. The method of claim 1, wherein the diagnostic result includes internal organ position information indicating that the stored file corresponds to the image data of a prescribed internal organ of the digestive organ, disease type information indicating that the stored file matches the image data of a predetermined disease and disease seriousness information indicating a seriousness level of the disease if the stored file matches the image data indicating the prescribed disease.

13. The method of claim 1, wherein the image tool comprises an icon that indicates the attribute information by displaying a shape, color and color thickness of the icon differently.

14. The method of claim 1, wherein an additional image tool indicating a count of the image tool and other image tools indicating the same attribute information of the image tool is further created and then displayed on the screen.

15. The method of claim 14, wherein if the additional image tool is selected, at least one file having the same attribute information of the image tool are simultaneously displayed on the screen together with the stored file.

16. The method of claim 1, wherein if the image tools is selected, a window for displaying the stored file is provided to the screen by varying a rim color of the window according to the attribute information of the file.

17. The method of claim 16, wherein time attribute of the stored file is displayed below the window by varying the rim color of the window to discriminate whether the file is a file of the image data captured at a position of which internal organ of a digestive organ.

18. The method of claim 1, further comprising the step of performing image editing of the file if the stored file is displayed.

19. The method of claim 1, further comprising the steps of:
in case of displaying the stored file, displaying a comment input window for executing a comment information input of the file; and
if the comment information is inputted to the comment input window, storing the inputted comment information by having the inputted comment information interconnected to the file.

20. A capsule endoscope system comprising:
a capsule endoscope generating image data by capturing interior of a digestive organ of a human body by migrating along the digestive organ, the capsule endoscope consecutively transmitting the generated image data using the human body as a conductor;
a receiver storing the image data by receiving the image data from a sensor detecting the image data via the human body; and
a workstation receiving the stored image data from the receiver, the workstation playing back the received image data, the workstation capturing a portion of the received image data, the workstation storing the captured image data by storing a diagnostic result from the captured image data as attribute information together with the captured image data, the workstation creating an image tool indicating the attribute information and displaying the created image tool on a screen.

21. The capsule endoscope system of claim 20, wherein if the displayed image tool is selected, the workstation reads the stored image data and the attribute information and then displays the read image data and the read attribute information on the screen.

22. The capsule endoscope system of claim 20, wherein the workstation provides a time bar indicating a temporal length of whole image data received from the capsule endoscope to the screen.

23. The capsule endoscope system of claim 22, the time bar comprising:
a first indicator indicating a current playback position if the whole image data received from the capsule endoscope are played back; and
a second indicator indicating a view of the image data captured at a point where an internal organ of the digestive organ is changed in the whole image data.

24. The capsule endoscope system of claim 23, wherein if the second indicator is selected, the workstation displays an image frame (frame n) corresponding to the second indicator on the screen and simultaneously displays a plurality of image frames before the image frame (frame n) and a plurality of image frames after the image frame (frame n) among image frames of the whole image data received from the capsule endoscope on the screen as well.

25. The capsule endoscope system of claim 24, wherein the workstation displays a plurality of the image frames before the image frame (frame n) and a plurality of the image frames after the image frame (frame n) are arranged in order of timing centering on the image frame (frame n).

26. The capsule endoscope system of claim 24, wherein the workstation enlarges the image frame (frame n) and then displays the enlarged image frame.

27. The capsule endoscope system of claim 24, wherein the workstation enlarges the image frame (frame n), the image frame (frame n−1) right before the image frame (frame n) and the image frame (frame n+1) right after the image frame (frame n) and then displays the enlarged image frames.

28. The capsule endoscope system of claim 20, wherein the workstation provides an editing window for image editing of the captured image data to the screen.

29. The capsule endoscope system of claim 20, wherein the workstation provides a popup window for displaying an image indicating whether the captured image data is image data captured at which internal organ position of the digestive organ.

30. The capsule endoscope system of claim 20, wherein the workstation provides a popup window for displaying an image of the captured image data and an image previously stored for a disease symptom comparison.

31. The capsule endoscope system of claim 20, wherein the workstation provides a popup window including a list containing a per-internal organ position to select an internal organ position indicting whether the captured image data corresponds to the image data of a prescribed internal organ of the digestive organ, a list containing disease types to select whether the captured image data corresponds to the image data of a prescribed disease, and a list containing a level indicating seriousness of a disease if the captured image data is the image data indicating the disease.

32. The capsule endoscope system of claim 20, wherein the workstation provides the screen with a comment input window for performing a comment information input for the captured image data and wherein the workstation stores the comment information inputted via the comment input window by interconnecting the comment information to the captured image data.

33. The capsule endoscope system of claim 20, wherein the workstation sequentially arranges the received image data on a plurality of divided screens according to a time flow and then plays back the received image data simultaneously.

34. The capsule endoscope system of claim 20, wherein the image tool comprises an icon and represents the attribute information of the captured image data as a shape, color and color thickness of the icon.

35. The capsule endoscope system of claim 20, the workstation comprising:
a first workstation receiving the stored image data from the receiver, the first workstation capturing partial image data from the received image data, the first workstation transmitting the captured image data; and
a second workstation connected to the first workstation via a network, the second workstation receiving the captured image data from the first workstation, the second workstation storing the diagnostic result as the attribute information of the captured image data together with the received captured image data, the second workstation creating the image tool indicating the attribute information, the second workstation displaying the created image tool on the screen.

36. The capsule endoscope system of claim 35, wherein the second workstation provides a comment input window for performing a comment information input for the captured image data to the screen and wherein the second workstation stores the comment information inputted via the comment input window by interconnecting the comment information to the captured image data.
